(19) United States
(12) Patent Application Publication
(10) Pub. No.: US 2025/0146079 A1
(43) Pub. Date: May 8, 2025

(54) METHODS FOR DETECTING SURGICAL DRAIN FLUID EXOSOMES AND USES THEREOF

(71) Applicant: Washington University, St. Louis, MO (US)

(72) Inventors: Jose Zevallos, St. Louis, MO (US); Aadel Chaudhuri, St. Louis, MO (US)

(21) Appl. No.: 18/838,078

(22) PCT Filed: Feb. 14, 2023
(86) PCT No.: PCT/US2023/013014

§ 371 Date: Aug. 13, 2024

Related U.S. Application Data
(60) Provisional application No. 63/309,896, filed on Feb. 14, 2022.

(51) Int. Cl. C12Q 1/6886; C12Q 1/6806

(52) U.S. Cl. CPC C12Q 1/6886; C12Q 1/6806; C12Q 2600/118; C12Q 2600/158

(57) ABSTRACT

The present disclosure encompasses methods to detect. quantify. and/or analyze various surgical drain fluid (SDF) exosomal biomarkers and the uses thereof to inform diagnosis of diseases. select patients for further diagnostic testing, and guide treatment decisions.

### CROSS REFERENCE TO RELATED APPLICATIONS

This application claims the benefit of U.S. Provisional Patent Application Ser. No. 63/309,896, filed Feb. 14, 2022, the disclosure of which is hereby incorporated by reference in its entirety.

### GOVERNMENTAL RIGHTS

This work was supported by the U.S. Department of Veterans Affairs, and the Federal Government has certain rights in this invention.

### FIELD OF THE TECHNOLOGY

The present disclosure encompasses methods to detect, quantify, and/or analyze various biomarkers present in extracellular vesicles in surgical drain fluid (SDF) and uses thereof to inform diagnosis of diseases, select patients for further diagnostic testing, and guide treatment decisions.

### BACKGROUND

Exosomes are nano-sized bi-lipid membrane vesicles secreted from living cells, which play important functions in cell-cell communications. Exosomes contain active biologies including lipids, cytokines, microRNA, mRNA and DNA, as well as, proteins, which can be presented on the surface of the exosomes. Exosomes are thought to be useful for many therapeutic approaches including immune modulation, the promotion of angiogenesis, and for the delivery of medicaments. However, the pathophysiological role of exosomes in various disease remains unknown. Moreover, the usability of postoperative surgical drainage fluid as a source of isolating exomosmes and use thereof for diagnostic purposes has not be elucidated.

Accordingly, there remains a need in the art for improved methods to detect, analzye and exosomal-biomarkers present in surgical drain fluid.

### SUMMARY

One aspect of the present disclosure encompasses a method for detecting an exosome-associated biomarker in a biological sample. The method generally comprises isolating extracellular vesicles from a surgical drain fluid; and detecting at least one biomarker produced in the extracellular vesicles.

Another aspect of the present disclosure encompasses a method of measuring a treatment response in a subject having or at risk of having disease. Typically, the method comprises (a) quantifying, in a first SDF sample obtained from a subject, an exosome-associated biomarker; (b) administering a treatment to the subject; and (c) quantifying, in a SDF sample obtained from the subject after the treatment, the biomarker quantified in step (a); wherein no change or a decrease in the amount of the biomarker in the second sample, as compared to the first sample, indicates a positive treatment response, or wherein the amount of the biomarker increases in the second sample as compared to the first sample but the change is less than a change that occurs in a control group of subjects that have the disease but were not administered treatment.

Yet another aspect of the present disclosure encompasses a method of monitoring a subject having or at risk of having disease. The method usually comprises quantifying a biomarker in a first SDF sample obtained from the subject and a second SDF sample obtained from the subject, wherein the second biological sample was obtained after the first biological sample; wherein an increase in the amount of the biomarker in the second sample as compared to the first sample indicates an increase in disease.

Other aspects and iterations of the present disclosure are detailed below.

### DETAILED DESCRIPTION

Among the various aspects of the disclosure is the provision of methods to detect and optionally quantify biomarkers derived from extracellular vesicles present in surgical drain fluid and use of the methods to detect and optionally measure levels of those biomarkers as a disease diagnostic. As described in greater detail herein, it has been discovered that certain exosomal biomarkers in surgical drain fluid are indicators of disease, treatment efficacy and/or recurrance. The extracellular vesicles may be any membrane-bounce vesicle and preferably include exosomes, apoptotic bodies and microvesicles.

### I. Methods for Detecting Exosome-associated Biomarkers in a Surgical Drain Fluid

In one aspect aspect, the present disclosure provides methods for detecting extravesicular biomoarkers in a surgical drain fluid sample. Preferred methods comprises isolating extracelluar vesicles from a biological sample, and detecting associated biomarkers in the sample that are derived from the extracellular vesicles.

Suitable biological samples include a surgical drain fluid sample obtained from a subject.

The size of the biological sample used may vary depending upon the sample type, the health status of the subject from whom the sample was obtained, and the exosomal analytes to be analyzed. Surgical draining fluid sample volumes may be about 0.01 mL to about 5 mL, or about 0.05 mL to about 5 mL. In a specific example, the size of the sample may be about 0.05 mL to about 1 mL SDF. In some embodiments, SDF sample volumes may be about 0.01 mL to about 20 mL, or about 0.1 mL to about 20 mL. In a specific example, the size of the sample may be about 1 mL to about 20 mL SDF.

In some embodiments, the subject is a human. A human subject may be waiting for medical care or treatment, may be under medical care or treatment, or may have received medical care or treatment. In various embodiments, a human subject may be a healthy subject, a subject at risk of developing a disease or a subject having a disease.

In some embodiments the disease is cancer or precancer. Non-limiting examples of cancer cells include oropharyngeal cancer cells, lung cancer cells, breast cancer cells, melanoma cells, colon cancer cells, thyroid cancer cells, prostate cancer cells, ovarian cancer cells, testicular cancer cells, penile cancer cells, cervical cancer cells, anal cancer cells, brain cancer cells, liver cancer cells, pancreatic cancer cells, and testicular cancer cells.

Additional non-limiting examples of cancer cells include cells from a variety of cancer types including Acute Lymphoblastic Leukemia (ALL); Acute Myeloid Leukemia (AML); Adrenocortical Carcinoma; AIDS-Related Cancers; Kaposi Sarcoma (Soft Tissue Sarcoma); AIDS-Related Lymphoma (Lymphoma); Primary CNS Lymphoma (Lymphoma); Anal Cancer; Appendix Cancer; Gastrointestinal Carcinoid Tumors; Astrocytomas; Atypical Teratoid/Rhabdoid Tumor, Childhood, Central Nervous System (Brain Cancer); Basal Cell Carcinoma of the Skin; Bile Duct Cancer; Bladder Cancer; Bone Cancer (including Ewing Sarcoma and Osteosarcoma and Malignant Fibrous Histiocytoma); Brain Tumors; Breast Cancer; Bronchial Tumors; Burkitt Lymphoma; Carcinoid Tumor (Gastrointestinal); Childhood Carcinoid Tumors; Cardiac (Heart) Tumors; Central Nervous System cancer; Atypical Teratoid/Rhabdoid Tumor, Childhood (Brain Cancer); Embryonal Tumors, Childhood (Brain Cancer); Germ Cell Tumor, Childhood (Brain Cancer); Primary CNS Lymphoma; Cervical Cancer; Cholangiocarcinoma; Bile Duct Cancer Chordoma; Chronic Lymphocytic Leukemia (CLL); Chronic Myelogenous Leukemia (CML); Chronic Myeloproliferative Neoplasms; Colorectal Cancer; Craniopharyngioma (Brain Cancer); Cutaneous T-Cell; Ductal Carcinoma In Situ (DCIS); Embryonal Tumors, Central Nervous System, Childhood (Brain Cancer); Endometrial Cancer (Uterine Cancer); Ependymoma, Childhood (Brain Cancer); Esophageal Cancer; Esthesioneuroblastoma; Ewing Sarcoma (Bone Cancer); Extracranial Germ Cell Tumor; Extragonadal Germ Cell Tumor; Eye Cancer; Intraocular Melanoma; Intraocular Melanoma; Retinoblastoma; Fallopian Tube Cancer; Fibrous Histiocytoma of Bone, Malignant, or Osteosarcoma; Gallbladder Cancer; Gastric (Stomach) Cancer; Gastrointestinal Carcinoid Tumor; Gastrointestinal Stromal Tumors (GIST) (Soft Tissue Sarcoma); Germ Cell Tumors; Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease; Hairy Cell Leukemia; Head and Neck Cancer; Heart Tumors; Hepatocellular (Liver) Cancer; Histiocytosis, Langerhans Cell; Hodgkin Lymphoma; Hypopharyngeal Cancer; Intraocular Melanoma; Islet Cell Tumors; Pancreatic Neuroendocrine Tumors; Kaposi Sarcoma (Soft Tissue Sarcoma); Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small cell); Lymphoma; Male Breast Cancer; Malignant Fibrous Histiocytoma of Bone or Osteosarcoma; Melanoma; Melanoma, Intraocular (Eye); Merkel Cell Carcinoma (Skin Cancer); Mesothelioma, Malignant; Metastatic Cancer; Metastatic Squamous Neck Cancer with Occult Primary; Midline Tract Carcinoma Involving NUT Gene; Mouth Cancer; Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms; Nasal Cavity and Paranasal Sinus Cancer; Nasopharyngeal Cancer; Neuroblastoma; NonHodgkin Lymphoma; Non-Small Cell Lung Cancer; Oral Cancer, Lip or Oral Cavity Cancer; Oropharyngeal Cancer; Osteosarcoma and Malignant Fibrous Histiocytoma of Bone; Ovarian Cancer Pancreatic Cancer; Pancreatic Neuroendocrine Tumors (Islet Cell Tumors); Papillomatosis; Paraganglioma; Paranasal Sinus and Nasal Cavity Cancer; Parathyroid Cancer; Penile Cancer; Pharyngeal Cancer; Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer Renal Cell (Kidney) Cancer; Retinoblastoma; Rhabdomyosarcoma, Childhood (Soft Tissue Sarcoma); Salivary Gland Cancer; Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Uterine Sarcoma; Sézary Syndrome (Lymphoma); Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; T-Cell Lymphoma, Cutaneous; Lymphoma; Mycosis Fungoides and Sèzary Syndrome; Testicular Cancer; Throat Cancer; Nasopharyngeal Cancer; Oropharyngeal Cancer; Hypopharyngeal Cancer; Thymoma and Thymic Carcinoma; Thyroid Cancer; Thyroid Tumors; Transitional Cell Cancer of the Renal Pelvis and Ureter (Kidney (Renal Cell) Cancer); Ureter and Renal Pelvis; Transitional Cell Cancer (Kidney (Renal Cell) Cancer; Urethral Cancer; Uterine Cancer, Endometrial; Uterine Sarcoma; Vaginal Cancer; Vascular Tumors (Soft Tissue Sarcoma); Vulvar Cancer; or Wilms Tumor.

In some embodiments the disease is a lung disease. In some embodiments the lung disease is selected from the group consisting of acute lung injury, acute and chronic diseases, asthma, chronic obstructive pulmonary disease (COPD), lung fibrosis, idiopathic pulmonary fibrosis, recovery of lung surgery after lung cancer, pulmonary embolism, acute respiratory distress syndrome, pneumonia, viral infection, coronavirus infection, Covid-19, and ventilator induced lung injury.

In some embodiments the disease is a liver disease. In some embodiments the liver disease is selected from the group consisting of acute liver injury, acute and chronic diseases, liver cirrhosis, liver fibrosis, liver inflammation, metabolic disorders, liver damages caused by drugs, poisons, alcohol, virus (e.g., hepatitis) or other infectious disease, and cholestatic liver diseases.

In some embodiments the disease a brain/spinal cord disease. In some embodiments the brain/spinal cord disease is selected from the group consisting of acute brain/spinal cord injury, acute and chronic diseases, stroke, transient ischemic attach, Parkinson's and other movement disorders, dementias, Alzheimer's diseases epilepsy/seizures, myelopathy, multiple sclerosis, infections of the central nervous system, spinal cord trauma, spinal cord inflammation, amyotrophic lateral sclerosis, spinal muscular atrophy. In some embodiments, the brain/spinal cord disease is a neurodegenerative disease. The neurodegenerative disease may be amyotrophic lateral sclerosis, Charcot-Marie-Tooth disease, chronic traumatic encephalopathy (CTE), Creutzfeldt-Jacob disease, Dementia pugilistica, Down's Syndrome, Gerstmann-Sträussler-Scheinker disease, Huntington's disease, inclusion-body myositis, prion protein cerebral amyloid angiopathy, traumatic brain injury (TBI), amyotrophic lateral sclerosis/parkinsonism-dementia complex of Guam, Non-Guamanian motor neuron disease with neurofibrillary tangles, argyrophilic grain dementia, corticobasal degeneration, diffuse neurofibrillary tangles with calcification, frontotetemporal dementia, frontotemporal dementia with parkinsonism linked to chromosome 17, Hallevorden-Spatz disease, Lewy body dementia (LBD), multiple sclerosis, multiple system atrophy, Myotonic dystrophy, Niemann-Pick disease type C, Pallido-ponto-nigral degeneration, Parkinson's disease, Pick's disease, progressive subcortical gliosis, Postencephalitic Parkinsonism, PART (primary age-related Tauopathy), progressive supranuclear palsy, Subacute sclerosing panencephalitis, subacute sclerosis panencephalopathy, Tangle only dementia (or tangle predominant dementia), tangle predominant dementia, white matter tauopathy with globular glial inclusions, mild cognitive impairment (MCI), glaucoma, familial British dementia, familiar Danish dementia, Guadeloupean Parkinsonism, neurodegeneration with brain iron accumulation, SLC9A6-related mental retardation, HIV-related dementia, senile cardiac amyloidosis.

In some embodiments the disease is a kidney disease. In some embodiments the kidney disease is selected from the group consisting of acute kidney injury, acute and chronic diseases, kidney injury or damage induced by trauma, drugs (e.g., chemotherapeutic agents), kidney cysts, kidney stones, and kidney infections, recovery of kidney function after kidney transplant, diabetic nephropathy, and polycystic kidney disease.

In some embodiments the disease is a gastrointestinal disease. In some embodiments the gastrointestinal disease is selected from the group consisting of acute gastrointestinal injury, autoimmune disease, acute and chronic diseases, Crohn's disease, irritable bowel syndrome, perianal abscesses, colitis, colon polyps and cancer.

In some embodiments the disease is a bone marrow disease. In some embodiments the bone marrow disease is selected from the group consisting of acute and chronic diseases, anemia, leukopenia, thrombocytopenia aplastic anemia, myeloproliferative disorders, and stem cell transplantation.

In some embodiments the disease is an eye disease. In some embodiments the eye disease is selected from the group consisting of acute eye injury, chronic and acute eye diseases, dry-eye syndrome and diabetic retinopathy, and macular degeneration.

In some embodiments the disease is a spleen disease. In some embodiments the spleen disease disorder or condition is selected from the group consisting of acute spleen injury, chronic and acute spleen diseases, diseases associated with enlarged or de-regulated spleen functions, and lupus.

In some embodiments the disease is a skin disease. In some embodiments the skin disease is selected from the group consisting of acute skin injury, chronic and acute skin diseases, diabetic foot ulcer, wound due to chemical burn, fire bum, skin or tissue damage caused, e.g., by injury, disease or surgical procedures, hair loss, a hair follicle disease, disorder or condition, wrinkles, and reduced firmness.

In some embodiments the disease is an ischemic disease. In some embodiments the ischemic disease is selected from the group consisting of acute ischemic injury, chronic and acute ischemic diseases, ischemic heart disease, ischemic vascular disease, ischemic colitis, mesenteric ischemia, Brain ischemia (e.g., stroke), acute or chronic limb ischemia, cutaneous ischemia, ischemic kidney, and the promotion of angiogenesis in tissues or organs in need thereof.

In some embodiments the disease is a heart/cardiovascular disease. In some embodiments the heart/cardiovascular disease is selected from the group consisting of acute heart/cardiovascular injury, hypertension, atherosclerosis, myocardial infarction (MI), and chronic heart failure.

In some embodiments the disease is an aging associated disease. In some embodiments the ageing associated disease is selected from the group consisting of age related fragility, age related diabetics, Alzheimer's diseases; age related macular degeneration, age related hearing loss, age related memory loss, age related cognitive decline, age related dementia, age related nuclear cataract, age associated loss of function and other effects of ageing.

In some embodiments the disease is a systemic disease. In some embodiments the systemic disease is selected from the group consisting of acute and chronic diseases, graft versus host disease, and infections (e.g., ear infection).

A healthy subject, sometimes referred to as a “control subject” or a “healthy control”, minimally has no clinical signs or symptoms of disease and may also be “negative” for other clinical signs or symptoms of a disease.

In other embodiments, the subject is a laboratory animal. In a further embodiment, the subject is a laboratory animal genetically engineered to be a model of disease.

SDF may have been obtained by any known method including, but not limited to, capturing a surgical drainage tube associated with the surgery. Non-limiting examples include a Jackson-Pratt (JP) drain, a Penrose drain with a collection tube. In some aspects, the sample may be obtained within about 24 hours of the completion of the surgery, providing the practitioner with timely information regarding disease-related genetic material in the surgical drainage that may be used to select additional treatments. In various aspects, the surgery includes any surgery directed at removing tissues from the subject including, but not limited to, resectioning surgery, dissection surgery, excision surgery, and any combination thereof.

Multiple SDF samples contemporaneously collected from a subject may be pooled to create “a sample”. Once collected, SDF samples may have been processed according to methods known in the art (e.g., centrifugation to remove whole cells and cellular debris; use of additives designed to stabilize and preserve the specimen prior to analytical testing; etc.). SDF samples may be used immediately or may be frozen and stored indefinitely.

Prior to use in the methods disclosed herein, a biological sample may also have been modified, if needed or desired, to include protease inhibitors, internal standards, detergent(s) and chaotropic agent(s), to deplete or enrich for other analytes (e.g. proteins, peptides, metabolites, etc.), or any combination thereof.

### Isolating one to a Plurality of Exosomes in the Biological Sample

In one aspect, the methods include isolating extracelllar vesicles (e.g., exosomes) from the SDF sample. In a non-limiting example, the surgical drainage fluid is centrifuged and filtered. EDTA is added to the sample to inhibit nucleases in the sample. The sample with added EDTA is further centrifuged, and the supernatant is removed and retained. In some aspects, the supernatant may be used as is for detection and quantification of cell-free DNA, RNA, and proteins. In other aspects, exosomes may be isolated from the supernatant by contacting the supernatant with a chromatographic media followed by elution. In various aspects, the exosomal isolation method is not particularly limited as long as the method yields intact exosomes. For example, some isolation methods, such as ultracentrification, may damage the exosomes.

The exosomes described herein contain markers that can be used to identify and/or isolate said exosomes. These markers may, for example, be proteins, nucleic acids, saccharide molecules, glycosylated proteins, lipid molecules, and may exist in monomeric, oligomeric and/or multimeric form. In certain embodiments, the markers are produced by the cell from which the exosomes are derived. In certain embodiments, the marker is provided by the cell from which the exosomes are derived, but the marker is not expressed at a higher level by said cell.

The three-dimensional structure of exosomes allows for the retention of markers on the surface of the exosome and/or contained within the exosome. Similarly, marker molecules may exist partially within the exosome, partially on the outer surface of the exosome and/or across the phospholipid bilayer of the exosome. In a specific embodiment, the markers associated with the exosomes described herein are proteins. In certain embodiments, the markers are transmembrane proteins that are anchored within the exosome phospholipid bilayer, or are anchored across the exosome phospholipid bilayer such that portions of the protein molecule are within the exosome while portions of the same molecule are exposed to the outer surface of the exosome. In certain embodiments, the markers are contained entirely within the exosome. In another specific embodiment, the markers associated with the exosomes described herein are nucleic acids. In certain embodiments, said nucleic acids are HPV DNA.

The exosomes described herein comprise surface markers that allow for their identification and that can be used to isolate/obtain substantially pure populations of cell exosomes free from their cells of origin and other cellular and non-cellular material. Methods of for determining exosome surface marker composition are known in the art. For example, exosomal surface markers can be detected by fluorescence-activated cell sorting (FACS) or Western blotting.

The exosomes described herein may be isolated in accordance with the methods described herein and their yields may be quantified. In a specific embodiment, the exosomes described herein are isolated at a concentration of about 0.5-5.0 mg per liter of sample. In another specific embodiment, the exosomes described herein are isolated at a concentration of about 2-3 mg per liter of culture medium (e.g, culture medium containing serum). In another specific embodiment, the exosomes described herein are isolated at a concentration of about 0.5-1.5 mg per liter of culture medium (e.g, culture medium lacking serum).

The exosomes described herein can be preserved, that is, placed under conditions that allow for long-term storage, or conditions that inhibit degradation of the exosomes.

In certain embodiments, the exosomes described herein can be stored after collection according to a method described above in a composition comprising a buffering agent at an appropriate temperature. In certain embodiments, the exosomes described herein are stored frozen, e.g, at about −20° C. or about −80° C.

In certain embodiments, the exosomes described herein can be cryopreserved, e.g, in small containers, e.g, ampoules (for example, 2 mL vials). In certain embodiments, the exosomes described herein are cryopreserved at a concentration of about 0.1 mg/mL to about 10 mg/mL.

In certain embodiments, the exosomes described herein are cryopreserved at a temperature from about −80°° C. to about −180° C.

Cryopreserved exosomes can be transferred to liquid nitrogen prior to thawing for use. In some embodiments, for example, once the ampoules have reached about −90° C., they are transferred to a liquid nitrogen storage area.

Cryopreservation can also be done using a controlled-rate freezer. Cryopreserved exosomes can be thawed at a temperature of about 25° C. to about 40° C. before use.

In certain embodiments, the exosomes described herein are stored at temperatures of about 4° C. to about 20° C. for short periods of time (e.g, less than two weeks).

### Detecting one to a Plurality of Biomarkers From Isolated Exosomes

Biomarkers can be detected, and optionally quantified, in the isolated exosome samples by mass spectrometry, as further detailed below or in the Examples, or by other methods known in the art, including but not limited to an immunoassay, a multiplexed assay (such as xMAP technology by Luminex), a single molecule array assay (such as Simoa® bead technology), a proximity ligation assay (such as DuoLink® by Sigma Aldrich), next generation DNA sequencing, next generation RNA sequencing, next generation protein sequencing, PCR, or the like.

### II. Methods for Detecting a Biomarker and use Thereof

In another aspect, the present disclosure provides a method for detecting, and optionally quantifying, a biomarker in a sample obtained from a subject. The method comprises detecting and optionally quantifying and exosome biomarker according to a method detailed above, wherein the biological sample is a sample obtained from a subject having or at risk of having disease, and wherein the biomarker is specific to the disease.

In some embodiments, the biomarker may be exosome associated HPV DNA. In one aspect, the HPV DNA is associated with oropharyngeal cancer cells. In some aspects, the methods include detecting and quantifying at least one HPV strain associated with HPV (+), non-limiting examples included HPV16 DNA, HPV18 DNA, HPV31 DNA, HPV33 fragment, HPV35 fragment, HPV45 DNA, HPV52 DNA, HPV58 DNA, and any combination thereof.

Detection and quantification of the biomarker may be used for a number of purposes. Non-limiting examples include diagnosing a disease, monitoring/measuring the development or progression of a disease state, treating a subject with a disease, determining/measuring the efficacy of a given treatment, and the like.

Accordingly, in another aspect, the method comprises detecting and quantifying the level of a biomarker, as described in any of the embodiments above, and determining if the level is present, absent, reduced, elevated or the same in comparison to its level in control subjects. In another aspect, the method comprises detecting and quantifying the level of a biomarker, as described in any of the embodiments above, and determining if the level is present, absent, reduced, elevated or the same in comparison to its level in control subjects who are cognitively normal and/or negative for one or more additional clinical sign or symptom of the disease or injury. Clinical tests for evaluating disease presence, impairment, symptoms, are known in the art.

In some embodiments, a subject may be diagnosed as having disease when the level of the biomarker significantly deviates from the mean in the control subjects. “Significantly deviates from the mean” refers to values that are at least 1 standard deviation, preferably at least 1.3 standard deviations, more preferably at least 1.5 standard deviations or even more preferably at least 2 standard deviations, above or below the mean (e.g., 10, 1.10, 1.20, 1.30, 1.40, 1.50, etc., where σ is the standard deviation defined by the normal distribution measured in a control population). In addition to using a threshold (e.g., at least 1 standard deviation above or below the mean), in some embodiments the extent of change above or below the mean may be used to diagnose a subject.

In another aspect, the method comprises detecting and quantifying the level of a biomarker, as described in any of the embodiments above, in a first biological sample obtained from the subject and a second biological sample obtained from the subject, wherein the first biological sample and the second biological sample are both a SDF sample, and wherein the second biological sample was obtained after the first biological sample.

In another aspect, the method comprises detecting and quantifying the level of a biomarker, as described in any of the embodiments above, in a first biological sample obtained from the subject and a second biological sample obtained from the subject, wherein the first biological sample and the second biological sample are both a SDF sample, and wherein the second biological sample was obtained after the first biological sample. An increase in the level of biomarker in the second sample as compared to the first sample indicates an increase in disease state or progression. In some embodiments, the rate of change in the levels of the biomarker between the first and subsequent samples is used to determine the stage of disease. Accordingly, such methods may be used to monitor a subject has disease or is at risk of having disease.

In some embodiments, one or more of the above methods may be used in combination with one or more disease biomarker known in the art to diagnose, stage, and/or treat specific neurodegenerative diseases.

In some embodiments, one or more of the above methods may be used determine whether a subject should receive additional diagnostic testing, which may, for instance, be a more invasive diagnostic method.

In some embodiments, one or more of the above methods may also be used to prognose disease status or disease progression.

In another aspect, the method comprises (a) detecting and quantifying the level of a biomarker, as described in any of the embodiments above, in a first biological sample obtained from the subject, a biomarker as described herein; (b) administering a treatment to the subject; and (c) detecting and quantifying, in a second biological sample obtained from the subject after the treatment, the biomarker quantified in step (a); wherein the first biological sample and the second biological sample are both SDF sample. Either no change in the level of the biomarker, or a decrease in the level of the biomarker, in the second sample as compared to the first sample indicates a positive treatment response. In addition, an increase in the level of the biomarker in the second sample as compared to the first sample may also indicate a positive treatment response when the increase is less than an increase that occurs in a control group of subjects that have disease but were not administered treatment. Preferably the control subjects have the same disease process or type of injury. Accordingly, such methods may be used to measure a treatment response in a subject having or at risk of having neuronal damage.

In another aspect, the present disclosure comprises treating a subject diagnosed with a disease. The method comprises (a) quantifying, in a sample obtained from the subject, a biomarker as described herein; and (b) administering to the subject a pharmaceutical composition to decrease or stabilize the amount of the biomarker measured in step (a).

### EXAMPLES

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying examples and drawings is to be interpreted as illustrative and not in a limiting sense.

### Example 1

Background: Human papillomavirus (HPV) is one of the main causes of oropharyngeal squamous cell carcinoma (OPSCC). Exosomes are nano-sized membrane vesicles of endocytic origin carrying DNA and RNA, and they exist in most body fluids like serum and urine. However, the pathophysiological role of exosomes in HPV+ OPSCC remains unknown. It was hypothesized that exosomes derived from postoperative surgical drainage fluid (SDF) of OPSCC contain HPV DNAs. It was further sought to determine the potential to exchange HPV DNA via exosomes in neighboring HPV negative cells.

Methods: Twenty-one HPV+ SDF (5 ml) and 5 HPV-SDF (5 ml) were collected after surgery in 26 OPSCC patients. Exosomes were isolated from SDF by using Qiagen exoEasy kit. Exosome structure was characterized by Transmission Electron Microscopy (TEM) and Nanoparticle Tracking Analysis (NTA). Western Blot (WB) was performed to verify the exosomal biomarkers, CD63, CD9 and TSG101. Exosomes were pretreated with DNase (New England Biolabs) to cleave the surface DNA, exosomal DNA was subsequently isolated by using Sigma cell-free DNA isolation kit. TaqMan real-time PCR was employed to detect the copy number of HPV16 DNA. Exosomes isolated from 3 HPV+ SDF and 1 HPV-SDF were used to incubate with HPV-cell lines, SCC22A and SCC25, to examine the shuttle potential.

Results: TEM demonstrated that exosomes were round or oval shaped vesicles in SDF (70-150 nm). NTA analysis demonstrated that there were 1.51±1.44×10¹¹ particles/ml of microvesicles in SDF. WB demonstrated that CD63, CD9, and TSG101 proteins were detected in SDF derived exosomes. TaqMan PCR demonstrated that HPV DNA was successfully detected in exosome DNA isolated from SDF of 80.9% (n=17) HPV+ OPSCC patients. Exosomes isolated from HPV-patients (n=5) SDF shown undetectable of HPV16 DNA. Quantification of HPV16 copy number demonstrated that there were 4.55±2.31 (log10) HPV16 copies/ml in SDF cfDNA, and 4.48±1.97 (log10) HPV16 copies/ml in exosomes (Statistical comparison), indicating that the majority of circulating HPV16 DNA resides in exosomes not naked DNA in SDF. An in vitro cell culture assay shown that exosomal HPV16 DNA can be uptaken and transferred into SCC22A and SCC25 cells.

Conclusions: This is the first study to demonstrate exosomes are present in SDF in OPSCC patients. A majority of circulating HPV16 DNA is found to be localized in exosomes in drain fluid of HPV+ OPSCC patients. Functionally, HPV16 DNA can actively shuttle between neighboring cells, which provide the evidence that re-infection of HPV via exosomes would enhance tumor recurrence after surgery or tumor dormancy.

What is claimed is:

1. A method for detecting an exosome-associated biomarker in a biological sample, the method comprising
isolating extracellular vesicles from a surgical drain fluid; and
detecting at least one biomarker produced in the extracellular vesicles.

2. The method of claim 1, wherein the isolating step comprises
filtering and centrifuging the drain fluid, and contacting the drain fluid with
a chromatography medium.

3. The method of claim 1, wherein the surgical drain fluid is captured from a resectioning surgery, a dissection surgery, an excision surgery, and any combination thereof.

4. The method of claim 1, wherein the biomarker is one or more of a protein, nucleic acid, saccharide molecule, glycosylated protein, lipid molecule, and may exist in monomeric, oligomeric and/or multimeric form.

5. The method of claim 4, wherein the extracellular vesicles are exosomes, apoptotic bodies, or microvesicles.

6. The method of claim 4 or claim 5, wherein the presence, absence, or differential expression of the biomarkers relative to a healthy control is an indication of disease, disease progression, or treatment efficacy.

7. The method of claim 6, wherein the disease is cancer.

8. The method of claim 7, wherein the cancer is oropharyngeal cancer, lung cancer, breast cancer, melanoma, colon cancer, thyroid cancer, prostate cancer, ovarian cancer, testicular cancer, penile cancer, cervical cancer, anal cancer, brain cancer, liver cancer, pancreatic cancer, or testicular cancer.

9. The method of claim 7, wherein the cancer is Acute Lymphoblastic Leukemia (ALL); Acute Myeloid Leukemia (AML); Adrenocortical Carcinoma; AIDS-Related Cancers; Kaposi Sarcoma (Soft Tissue Sarcoma); AIDS-Related Lymphoma (Lymphoma); Primary CNS Lymphoma (Lymphoma); Anal Cancer; Appendix Cancer; Gastrointestinal Carcinoid Tumors; Astrocytomas; Atypical Teratoid/Rhabdoid Tumor, Childhood, Central Nervous System (Brain Cancer); Basal Cell Carcinoma of the Skin; Bile Duct Cancer; Bladder Cancer; Bone Cancer (including Ewing Sarcoma and Osteosarcoma and Malignant Fibrous Histiocytoma); Brain Tumors; Breast Cancer; Bronchial Tumors; Burkitt Lymphoma; Carcinoid Tumor (Gastrointestinal); Childhood Carcinoid Tumors; Cardiac (Heart) Tumors; Central Nervous System cancer; Atypical Teratoid/Rhabdoid Tumor, Childhood (Brain Cancer); Embryonal Tumors, Childhood (Brain Cancer); Germ Cell Tumor, Childhood (Brain Cancer); Primary CNS Lymphoma; Cervical Cancer; Cholangiocarcinoma; Bile Duct Cancer Chordoma; Chronic Lymphocytic Leukemia (CLL); Chronic Myelogenous Leukemia (CML); Chronic Myeloproliferative Neoplasms; Colorectal Cancer; Craniopharyngioma (Brain Cancer); Cutaneous T-Cell; Ductal Carcinoma In Situ (DCIS); Embryonal Tumors, Central Nervous System, Childhood (Brain Cancer); Endometrial Cancer (Uterine Cancer); Ependymoma, Childhood (Brain Cancer); Esophageal Cancer; Esthesioneuroblastoma; Ewing Sarcoma (Bone Cancer); Extracranial Germ Cell Tumor; Extragonadal Germ Cell Tumor; Eye Cancer; Intraocular Melanoma; Intraocular Melanoma; Retinoblastoma; Fallopian Tube Cancer; Fibrous Histiocytoma of Bone, Malignant, or Osteosarcoma; Gallbladder Cancer; Gastric (Stomach) Cancer; Gastrointestinal Carcinoid Tumor; Gastrointestinal Stromal Tumors (GIST) (Soft Tissue Sarcoma); Germ Cell Tumors; Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease; Hairy Cell Leukemia; Head and Neck Cancer; Heart Tumors; Hepatocellular (Liver) Cancer; Histiocytosis, Langerhans Cell; Hodgkin Lymphoma; Hypopharyngeal Cancer; Intraocular Melanoma; Islet Cell Tumors; Pancreatic Neuroendocrine Tumors; Kaposi Sarcoma (Soft Tissue Sarcoma); Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small cell); Lymphoma; Male Breast Cancer; Malignant Fibrous Histiocytoma of Bone or Osteosarcoma; Melanoma; Melanoma, Intraocular (Eye); Merkel Cell Carcinoma (Skin Cancer); Mesothelioma, Malignant; Metastatic Cancer; Metastatic Squamous Neck Cancer with Occult Primary; Midline Tract Carcinoma Involving NUT Gene; Mouth Cancer; Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms; Nasal Cavity and Paranasal Sinus Cancer; Nasopharyngeal Cancer; Neuroblastoma; NonHodgkin Lymphoma; Non-Small Cell Lung Cancer; Oral Cancer, Lip or Oral Cavity Cancer; Oropharyngeal Cancer; Osteosarcoma and Malignant Fibrous Histiocytoma of Bone; Ovarian Cancer Pancreatic Cancer; Pancreatic Neuroendocrine Tumors (Islet Cell Tumors); Papillomatosis; Paraganglioma; Paranasal Sinus and Nasal Cavity Cancer; Parathyroid Cancer; Penile Cancer; Pharyngeal Cancer; Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer Renal Cell (Kidney) Cancer; Retinoblastoma; Rhabdomyosarcoma, Childhood (Soft Tissue Sarcoma); Salivary Gland Cancer; Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Uterine Sarcoma; Sézary Syndrome (Lymphoma); Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; T-Cell Lymphoma, Cutaneous; Lymphoma; Mycosis Fungoides and Sèzary Syndrome; Testicular Cancer; Throat Cancer; Nasopharyngeal Cancer; Oropharyngeal Cancer; Hypopharyngeal Cancer; Thymoma and Thymic Carcinoma; Thyroid Cancer; Thyroid Tumors; Transitional Cell Cancer of the Renal Pelvis and Ureter (Kidney (Renal Cell) Cancer); Ureter and Renal Pelvis; Transitional Cell Cancer (Kidney (Renal Cell) Cancer; Urethral Cancer; Uterine Cancer, Endometrial; Uterine Sarcoma; Vaginal Cancer; Vascular Tumors (Soft Tissue Sarcoma); Vulvar Cancer; or Wilms Tumor.

10. The method of claim 6, wherein the disease is a lung disease.

11. The method of claim 10, wherein the lung disease is selected from the group consisting of acute lung injury, acute and chronic diseases, asthma, chronic obstructive pulmonary disease (COPD), lung fibrosis, idiopathic pulmonary fibrosis, recovery of lung surgery after lung cancer, pulmonary embolism, acute respiratory distress syndrome, pneumonia, viral infection, coronavirus infection, Covid-19, and ventilator induced lung injury.

12. The method of claim 6, wherein the disease is a liver disease.

13. The method of claim 12, wherein the liver disease is selected from the group consisting of acute liver injury, acute and chronic diseases, liver cirrhosis, liver fibrosis, liver inflammation, metabolic disorders, liver damages caused by drugs, poisons, alcohol, virus (e.g., hepatitis) or other infectious disease, and cholestatic liver diseases.

14. The method of claim 6, wherein the disease a brain and/or spinal cord disease.

15. The method of claim 14, wherein in some embodiments the brain and/or spinal cord disease is selected from the group consisting of acute brain/spinal cord injury, acute and chronic diseases, stroke, transient ischemic attack, Parkinson's and other movement disorders, dementias, Alzheimer's diseases epilepsy/seizures, myelopathy, multiple sclerosis, infections of the central nervous system, spinal cord trauma, spinal cord inflammation, amyotrophic lateral sclerosis, spinal muscular atrophy, neurodegenerative disease, amyotrophic lateral sclerosis, Charcot-Marie-Tooth disease, chronic traumatic encephalopathy (CTE), Creutzfeldt-Jacob disease, Dementia pugilistica, Down's Syndrome, Gerstmann-Sträussler-Scheinker disease, Huntington's disease, inclusion-body myositis, prion protein cerebral amyloid angiopathy, traumatic brain injury (TBI), amyotrophic lateral sclerosis/parkinsonism-dementia complex of Guam, Non-Guamanian motor neuron disease with neurofibrillary tangles, argyrophilic grain dementia, corticobasal degeneration, diffuse neurofibrillary tangles with calcification, frontotetemporal dementia, frontotemporal dementia with parkinsonism linked to chromosome 17, Hallevorden-Spatz disease, Lewy body dementia (LBD), multiple sclerosis, multiple system atrophy, Myotonic dystrophy, Niemann-Pick disease type C, Pallido-ponto-nigral degeneration, Parkinson's disease, Pick's disease, progressive subcortical gliosis, Postencephalitic Parkinsonism, PART (primary age-related Tauopathy), progressive supranuclear palsy, Subacute sclerosing panencephalitis, subacute sclerosis panencephalopathy, Tangle only dementia (or tangle predominant dementia), tangle predominant dementia, white matter tauopathy with globular glial inclusions, mild cognitive impairment (MCI), glaucoma, familial British dementia, familiar Danish dementia, Guadeloupean Parkinsonism, neurodegeneration with brain iron accumulation, SLC9A6-related mental retardation, HIV-related dementia, and senile cardiac amyloidosis.

16. The method of claim 6, wherein the disease is a kidney disease.

17. The method of claim 16, wherein the kidney disease is selected from the group consisting of acute kidney injury, acute and chronic diseases, kidney injury or damage induced by trauma, drugs (e.g., chemotherapeutic agents), kidney cysts, kidney stones, and kidney infections, recovery of kidney function after kidney transplant, diabetic nephropathy, and polycystic kidney disease.

18. The method of claim 6, wherein the disease is a gastrointestinal disease.

19. The method of claim 18, wherein the gastrointestinal disease is selected from the group consisting of acute gastrointestinal injury, autoimmune disease, acute and chronic diseases, Crohn's disease, irritable bowel syndrome, perianal abscesses, colitis, colon polyps and cancer.

20. The method of claim 6, wherein the disease is a bone marrow disease.

21. The method of claim 20, wherein the bone marrow disease is selected from the group consisting of acute and chronic diseases, anemia, leukopenia, thrombocytopenia aplastic anemia, myeloproliferative disorders, and stem cell transplantation.

22. The method of claim 6, wherein the disease is an eye disease.

23. The method of claim 22, wherein the eye disease is selected from the group consisting of acute eye injury, chronic and acute eye diseases, dry-eye syndrome and diabetic retinopathy, and macular degeneration.

24. The method of claim 6, wherein the disease is a spleen disease.

25. The method of claim 24, wherein the spleen disease disorder or condition is selected from the group consisting of acute spleen injury, chronic and acute spleen diseases, diseases associated with enlarged or de-regulated spleen functions, and lupus.

26. The method of claim 6, wherein the disease is a skin disease.

27. The method of claim 26, wherein the skin disease is selected from the group consisting of acute skin injury, chronic and acute skin diseases, diabetic foot ulcer, wound due to chemical burn, fire bum, skin or tissue damage caused, e.g., by injury, disease or surgical procedures, hair loss, a hair follicle disease, disorder or condition, wrinkles, and reduced firmness.

28. The method of claim 6, wherein the disease is an ischemic disease.

29. The method of 28, wherein the ischemic disease is selected from the group consisting of acute ischemic injury, chronic and acute ischemic diseases, ischemic heart disease, ischemic vascular disease, ischemic colitis, mesenteric ischemia, Brain ischemia (e.g., stroke), acute or chronic limb ischemia, cutaneous ischemia, ischemic kidney, and the promotion of angiogenesis in tissues or organs in need thereof.

30. The method of claim 6, wherein the disease is a heart and/or cardiovascular disease.

31. The method of claim 30, wherein the heart and/or cardiovascular disease is selected from the group consisting of acute heart/cardiovascular injury, hypertension, atherosclerosis, myocardial infarction (MI), and chronic heart failure.

32. The method of claim 6, wherein the disease is an aging associated disease.

33. The method of claim 32, wherein the ageing associated disease is selected from the group consisting of age related fragility, age related diabetics, Alzheimer's diseases; age related macular degeneration, age related hearing loss, age related memory loss, age related cognitive decline, age related dementia, age related nuclear cataract, age associated loss of function and other effects of ageing.

34. The method of claim 6, wherein the biomarker is HPV DNA.

35. The method of claim 34, wherein the HPV DNA is HPV16 DNA, HPV18 DNA, HPV31 DNA, HPV33 DNA, HPV35 DNA, HPV45 DNA, HPV52 DNA, HPV58 DNA, and any combinations or fragments thereof.

36. A method of measuring a treatment response in a subject having or at risk of having disease, the method comprising
(a) quantifying, in a first SDF sample obtained from a subject, an exosome-associated biomarker;
(b) administering a treatment to the subject; and
(c) quantifying, in a SDF sample obtained from the subject after the treatment, the biomarker quantified in step (a);
wherein no change or a decrease in the amount of the biomarker in the second sample, as compared to the first sample, indicates a positive treatment response, or wherein the amount of the biomarker increases in the second sample as compared to the first sample but the change is less than a change that occurs in a control group of subjects that have the disease but were not administered treatment.

37. A method of monitoring a subject having or at risk of having disease, the method comprising quantifying a biomarker according to any one of the proceeding claims, in a first SDF sample obtained from the subject and a second SDF sample obtained from the subject, wherein the second biological sample was obtained after the first biological sample;
wherein an increase in the amount of the biomarker in the second sample as compared to the first sample indicates an increase in disease.

38. The method of any one of the preceeding claims, wherein an additional treatment is selected based on the quantity of the exosome-associated biomarker.

39. The method of claim 38, wherein the additional treatment is selected from radiotherapy, chemotherapy, follow-up surgery, active surveillance with imaging, administration of a therapeutic agent, and any combination thereof.
